(19) United States
(12) Patent Application Publication
(10) Pub. No.: US 2007/0083244 A1
(43) Pub. Date: Apr. 12, 2007

(54) PROCESS FOR TUNING AN EMI FILTER TO REDUCE THE AMOUNT OF HEAT GENERATED IN IMPLANTED LEAD WIRES DURING MEDICAL PROCEDURES SUCH AS MAGNETIC RESONANCE IMAGING

(75) Inventors: Robert A. Stevenson, Canyon Country, CA (US); Christine A. Frysz, Marriottsville, MD (US)

Correspondence Address: KELLY LOWRY & KELLEY, LLP, 6320 CANOGA AVENUE, SUITE 1650, WOODLAND HILLS, CA, 91367, US

(73) Assignee: GREATBATCH-SIERRA, INC., Carson City, NV (US)

(21) Appl. No.: 11/163,915

(22) Filed: Nov. 3, 2005

Related U.S. Application Data
(60) Provisional application No. 60/724,479, filed on Oct. 6, 2005.

(51) Int. Cl. A61N 1/00

(52) U.S. Cl. 607/37

(57) ABSTRACT

In EMI filter assemblies incorporating one or more passive filter elements including feedthrough capacitors and lossy ferrite inductors, a process is provided for tuning the various components to reduce the amount of heat generated in implanted lead wires during medical procedures such as magnetic resonance imaging. The process includes selection and testing of individual components and an interative process involving tradeoffs and subsequent testing prior to finalization of the feedthrough design.

### BACKGROUND OF THE INVENTION

This invention generally relates to EMI filter assemblies incorporating one or more passive filter elements including feedthrough capacitors and lossy ferrite inductors, or conventional inductors or the like. These EMI filter assemblies are typically used in active implantable medical devices (AIMDs), such as cardiac pacemakers, cardioverter defibrillators, neurostimulators and the like, for decoupling and shielding internal electronic components of the AIMD from undesirable electromagnetic interference (EMI) signals.

Compatibility of cardiac pacemakers, implantable defibrillators and other types of AIMDs with magnetic resonance imaging (MRI) and other types of hospital diagnostic equipment has become a major issue. If one goes to the websites of the major cardiac pacemaker manufacturers in the United States, which include St. Jude Medical, Medtronic and Guidant, one will see that the use of MRI is generally contra-indicated with pacemakers and implantable defibrillators. See also “Safety Aspects of Cardiac Pacemakers in Magnetic Resonance Imaging”, a dissertation submitted to the Swiss Federal Institute of Technology Zurich presented by Roger Christoph Luchinger. “Dielectric Properties of Biological Tissues: I. Literature Survey”, by C. Gabriel, S. Gabriel and E. Cortout; “Dielectric Properties of Biological Tissues: II. Measurements and the Frequency Range 0 Hz to 20 GHz”, by S. Gabriel, R. W. Lau and C. Gabriel; “Dielectric Properties of Biological Tissues: III. Parametric Models for the Dielectric Spectrum of Tissues”, by S. Gabriel, R. W. Lau and C. Gabriel; and “Advanced Engineering Electromagnetics, C. A. Balanis, Wiley, 1989, all of which are incorporated herein by reference.

However, an extensive review of the literature indicates that MRI is indeed often used with pacemaker patients. The safety and feasibility of MRI in patients with cardiac pacemakers is an issue of gaining significance. The effects of MRI on patients' pacemaker systems have only been analyzed retrospectively in some case reports. There are a number of papers that indicate that MRI on new generation pacemakers can be conducted up to 0.5 Tesla (T). MRI is one of medicine's most valuable diagnostic tools. An absolute contra-indication for pacemaker patients means that pacemaker and ICD wearers are excluded from MRI. This is particularly true of scans of the thorax and abdominal areas. Because of MRI's incredible value as a diagnostic tool for imaging organs and other body tissues, many physicians simply take the risk and go ahead and perform MRI on a pacemaker patient. The literature indicates a number of precautions that physicians should take in this case, including limiting the power of the MRI magnetic field, programming the pacemaker to fixed or asynchronous pacing mode (activation of the reed switch), and then careful reprogramming and evaluation of the pacemaker and patient after the procedure is complete. There have been reports of latent problems with cardiac pacemakers after an MRI procedure occurring many days later.

There are three types of electromagnetic fields used in an MRI unit. The first type is the main static magnetic field which is used to align protons in body tissue. The field strength varies from 0.5 to 1.5 Tesla in most of the currently available MRI units in clinical use. Some of the newer MRI system fields can go as high as 4 to 5 Tesla. This is about 100,000 times the magnetic field strength of the earth. A static magnetic field can induce powerful mechanical forces on any magnetic materials implanted within the patient. This would include certain components within the cardiac pacemaker itself and or lead wire systems. It is not likely (other than sudden system shut down) that the static MRI magnetic field can induce currents into the pacemaker lead wire system and hence into the pacemaker itself. It is a basic principle of physics that a magnetic field must either be time-varying as it cuts across the conductor, or the conductor itself must move within the magnetic field for currents to be induced. The lossy ferrite inductor or toroidal slab concept as described herein is not intended to provide protection against static magnetic fields such as those produced by magnetic resonance imaging.

The second type of field produced by magnetic resonance imaging is the pulsed RF field which is generated by the body coil or head coil. This is used to change the energy state of the protons and illicit MRI signals from tissue. The RF field is homogeneous in the central region and has two main components: (1) the magnetic field is circularly polarized in the actual plane; and (2) the electric field is related to the magnetic field by Maxwell's equations. In general, the RF field is switched on and off during measurements and usually has a frequency of 21 MHz to 64 MHz to 128 MHz depending upon the static magnetic field strength.

The third type of electromagnetic field is the time-varying magnetic gradient fields which are used for spatial localization. These change their strength along different orientations and operating frequencies on the order of 1 kHz. The vectors of the magnetic field gradients in the X, Y and Z directions are produced by three sets of orthogonally positioned coils and are switched on only during the measurements.

A particular concern is due to excessive currents which can be induced in implanted lead wires from a medical diagnostic procedure. A typical example would be excessive currents induced due to the radio frequency (RF) pulsed field of an MRI system. These excessive currents can cause heating of the lead wire through high power (I²R) loss or heating in tissue due to excessive current flowing through tissue itself. This situation is not limited solely to magnetic resonance imaging (MRI). There are a number of other medical diagnostic and/or therapy procedures that involve RF fields. This includes diathermy, electrical surgical knives, such as the Bovi knife, RF ablation and the like. Anytime an implanted lead wire system is exposed to high power RF fields, current can be induced in the lead wire system. This is due to three primary mechanisms, which include induced magnetic coupling through bounded loop areas, induced currents through antenna action, or induced currents or voltages from current circulating in body tissue which create associated voltage drops.

Exemplary prior art feedthrough capacitor EMI filters for implantable medical devices are described in U.S. Pat. Nos. 5,333,095; 5,905,627; 5,973,906; 4,424,551; 4,220,813; 5,531,003; 5,867,361; and 6,414,835, the contents of which are incorporated herein. Feedthrough capacitors are very desirable for EMI filters in that they provide a very low impedance over a very broad range of frequencies. The geometry of the feedthrough capacitor is such that it acts as a coaxial device and is relatively free of self-resonances. This is not true of conventional rectangular chip capacitors or any capacitors with a lead wire. In those cases, the series inductance self resonates with the capacitor thereby rendering the capacitor ineffective as an EMI filter above the self-resonant frequency (the capacitor literally becomes inductive). Accordingly, the feedthrough capacitor has become the mainstay EMI filter for all types of implantable medical devices, including pacemakers, ICDs, neurostimulators and the like. The feedthrough capacitor functions by providing a very low impedance to the housing or casing of the implantable medical device. In the case of a cardiac pacemaker, for example, the housing is typically of titanium. The titanium housing forms an equipotential surface which is a very effective electromagnetic shield. For example, when exposed to the radiation energy from a microwave oven, this shield effectively blocks, reflects and absorbs such high frequency energy. The feedthrough capacitor works in concert with this electromagnetic shield by decoupling EMI which is picked up by implanted lead wires and shunting that high frequency energy to the titanium or other conductive housing of the implantable medical device. By shunting such energy, it turns into harmless eddy currents and therefore is dissipated as a very low level of harmless heat. This prevents the EMI energy from reaching the sensitive internal electronic circuits of the AIMD which can cause permanent or temporary malfunction.

However, the presence of the EMI filtered feedthrough capacitor, by definition, reduces the input impedance of the implantable medical device. As an example, again consider the case of a cardiac pacemaker. Without the EMI filtered feedthrough capacitor the input impedance of the pacemaker might be as high as 10,000 ohms at MRI pulsed frequencies. By placing the feedthrough capacitor(s) at the point of lead wire ingress into and out of a cardiac pacemaker, the feedthrough capacitor itself determines the input impedance. This input impedance varies with frequency according with the following formula: X_{c}=½πfC. Where X_{c} is equal to the capacitive reactance in ohms, f is the frequency in hertz, and C is the capacitance in farads. A typical capacitor value that is used in prior art feedthrough capacitors for AIMDs is about 4000 picofarads.

By way of further explanation, we will consider the capacitor reactance of this particular capacitor in a 3-Tesla MRI system. A 3-Tesla MRI system has an RF pulse frequency of approximately 128 MHz. Therefore, the capacitive reactance equation becomes ½π(128×10⁶ Hz)(4000×10⁻¹²F) or X_{c}=0.31Ω. If one considers EMI filter protection only, the feedthrough capacitor desirably lowers the input impedance of the cardiac pacemaker from approximately 10,000 ohms all the way down to 0.31 ohms. This effectively shorts or decouples the high frequency EMI associated with the 128 MHz signal to the titanium housing thereby preventing it from getting into the sensitive internal electronic circuits of the AIMD. However, this situation presents a dilemma when the AIMD patient is exposed to medical device procedures, such as MRI. The very powerful RF fields of the MRI system induce voltages (electromotive forces—EMFs) into the implanted lead wire system. The presence of this very low input impedance to the cardiac pacemaker (0.31 Ω) causes very high currents to flow due to Ohms Law. This can cause overheating of the lead wire itself or it can cause excessive current to flow at the point of tissue interface, for example, between pacemaker Tip and Ring electrodes and through the myocardial tissue and, for example, the right ventricle.

Why not use a much lower value of feedthrough capacitor, for example, 400 picofarads? A 400-picofarad feedthrough capacitor would present a 3.1 ohm input impedance to the cardiac pacemaker. This would greatly reduce the current in the associated lead wire system. The problem is that this would make the AIMD vulnerable to high frequency emitters, such as closely held cellular telephones and similar devices that are found in the patient environment. In addition, there are a number of compliance standards for active implantable medical devices. This includes ANSI/AAMI/PC69 (in the United States), CENELEC 45502-2-1 (in Europe) and a pending ISO standard. The ISO Neurostimulator Committee is also considering a draft standard for EMI compliance. In other words, the AIMD manufacturer must provide a high degree of input filtering not only to make the patient safe from environmental emitters, but to also comply with various regulatory EMI standards.

Why must this be done with passive components, such as capacitors, inductors and resistors? The answer is that active electronic filters do not have enough dynamic range in general to stay linear in the presence of very large RF fields such as those produced by medical diagnostic equipment. This is particularly problematic as microchips have become smaller and more dense. It was not very many years ago that it was possible to buy 8-micron microchip technology. However, it is now very difficult to buy even 4-micron technology, with newer microchips in the submicron range. This has many positive aspects which allow microchips to be smaller and pack in more transistors into smaller spaces. However, an undesirable trade off to these ultrathin technologies is that they operate at lower voltages and become increasingly sensitive to a lack of dynamic range or what's known as a limitation on quiescent operating point. In the presence of extremely large RF fields, such as those produced by MRI, such active filters go into a non-linear region. This actually creates more EMI as the incoming EMI signal is distorted which produces many undesirable harmonics and demodulation products.

Accordingly, there is a need for passive EMI filters which provide a high degree of EMI filtering protection to the AIMD electronics while at the same time limiting the current in the implanted lead wire system. The present invention meets these needs and provides other related advantages.

### SUMMARY OF THE INVENTION

The present invention resides in a process for tuning an EMI filter for an active implantable medical device (AIMD), wherein the EMI filter has a capacitor and an inductor/resistor element. The novel process of the present invention comprises the steps of: (1) evaluating input impedance of the AIMD; (2) configuring the physical relationship of the capacitor and the inductor/resistor element of the EMI filter based on the evaluated input impedance of the AIMD; (3) iteratively selecting component values for the capacitor and the inductor/resistor elements of the EMI filter; and (4) analyzing the impedance characteristics of the selected components through circuit simulation to assess (a) whether the impedance of the EMI filter has been raised sufficiently to reduce undesirable RF currents that would flow during medical diagnostic procedures, and (b) if the AIMD is adequately protected against environmental emitters and complies with regulatory requirements. The novel process of the present invention further includes the steps of (5) building a prototype of the AIMD comprising an EMI filter having selected components that have been assessed to be acceptable; (6) testing the prototype to determine whether the impedance of the EMI filter has been raised sufficiently to reduce undesirable currents that would flow during medical diagnostic procedures; and (7) testing the prototype to determine if the AIMD is adequately protected against environmental emitters and complies with regulatory requirements.

The steps of iteratively selecting and analyzing may be repeated (a) if the impedance of the EMI filter has not been raised sufficiently to reduce undesirable currents that would flow during medical diagnostic procedures, or (b) if the AIMD does not adequately protect against environmental emitters or comply with regulatory requirements. Further, the configuring, iteratively selecting and analyzing steps may be repeated if the prototype fails either of the testing steps. The configuring step may include the step of utilizing an inductive/resistive element located at a point of lead wire ingress and egress from the AIMD followed by the capacitor, where the capacitance value of said capacitor is minimized to reduce RF currents in a lead wire system of the AIMD.

The novel process may further include the step of optimizing component values of the capacitor and the inductor/resistor elements of the EMI filter such that an acceptable level of attenuation is achieved with the lowest possible value of feedthrough capacitance.

The evaluating step may include the steps of utilizing a network analyzer, a sophisticated materials analyzer or spectrum analyzer to look back into the terminal of the AIMD where its implantable leads would normally connect, and performing impedance measurements at RF frequencies of interest.

One or more passive series inductive/resistive elements may be utilized to create a multi-element EMI filter having acceptable attenuation to protect a patient from electromagnetic interference (EMI). The one or more passive series elements may comprise an inductor, a resistor, a combined inductive/resistance element, an air wound inductor, a chip inductor, a wire wound resister, a composition resistor, or a toroidal or solenad inductor with a ferromagnetic material core. The passive series element may alternatively comprise a lead wire through the capacitor or a lossy ferrite conductor slab.

The capacitor and the passive series inductive and/or resistive elements may be combined to form an L, PI, T, LL, 5-element or N-element device.

Further, the iteratively selecting step includes the step of selecting a capacitor with a very low value of capacitance and selecting the maximum value of the inductor/resistor element that would physically fit the geometry available inside the package of the AIMD. The analyzing step may include the step of utilizing a network or spectrum analyzer to analyze the impedance of lead wire systems associated with the AIMD.

Other features and advantages of the present invention will become apparent from the following more detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 illustrates electrical schematics for several low pass filter EMI filter circuits;

FIG. 2 illustrates attenuation slope curves for various low pass filter circuits;

FIG. 3 is a schematic illustration of a human body illustrating various types of active implantable medical devices (AIMDs) currently in use;

FIG. 4 is a sectional view illustrating a quadpolar T circuit filter configuration;

FIG. 5 is a flow chart illustrating the prior art process of designing feedthrough capacitor filters to reduce or eliminate high frequency EMI from entering via implanted lead wires into the AIMD;

FIG. 6 is a flow chart illustrating the tuning process of the present invention;

FIG. 7 is a sectional view of a prior art unipolar feedthrough filter assembly;

FIG. 8 is a perspective and partially sectional view of the capacitor illustrated in FIG. 7;

FIG. 9 is an electrical schematic of the typical prior art feedthrough filter capacitor assembly of FIG. 7; and

FIG. 10 is an electrical schematic showing fine-tuning of the feedthrough assembly utilizing inductive differences in the lead wire.

### DETAILED DESCRIPTION OF THE PREFERRED EMBODIMENT

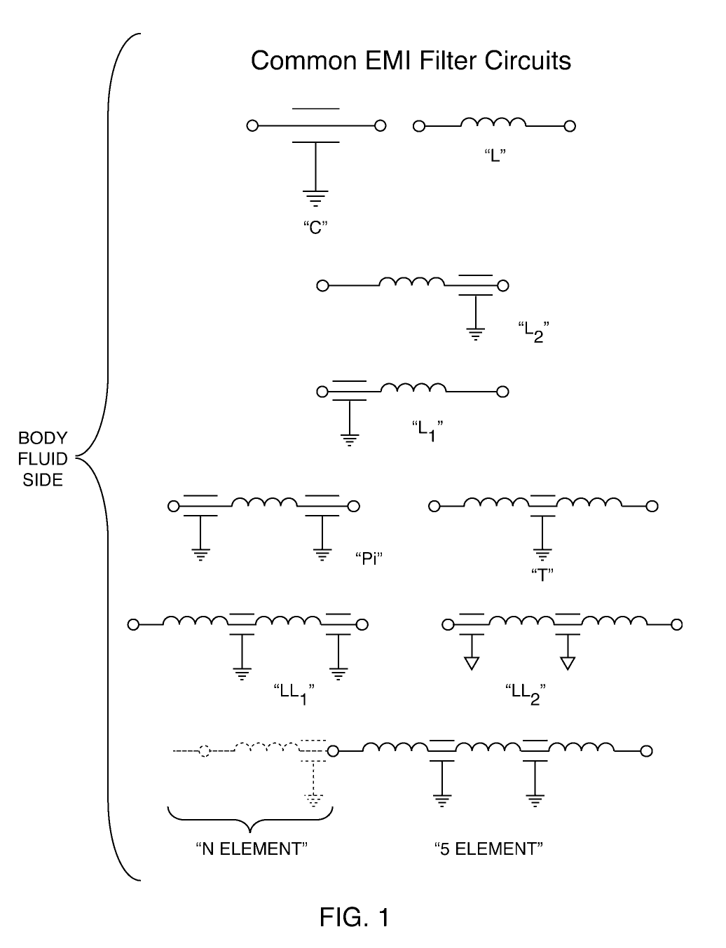

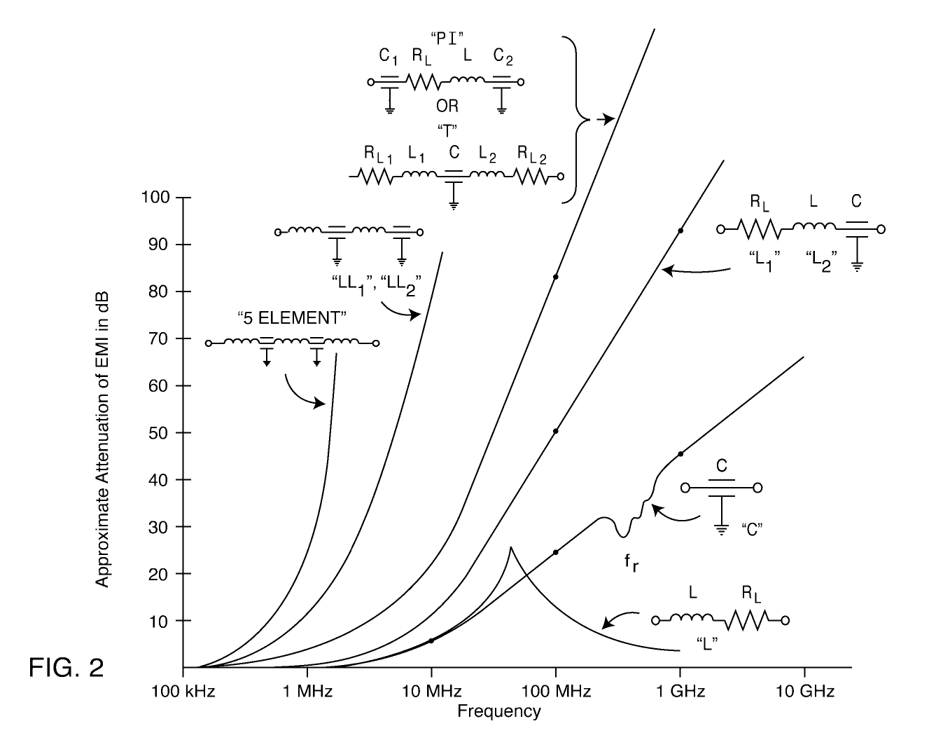

With reference to FIG. 2, the present invention requires a tuning or balancing of one or more feedthrough capacitors which are placed in series with one or more lossy (resistive) ferrite slab, inductor and/or resistive elements. The presence of the lossy ferrite slab and/or multiple turn inductor elements provides a series resistance and reactance. These series reactances tend to raise the AIMD input impedance. As previously described in the co-pending applications, the additional circuit elements also increase the attenuation slope of the EMI filter. This can be clearly seen in FIGS. 1 and 2. These figures are identical to FIGS. 20 and 21 of U.S. patent application Ser. No. 11/097,999, and similar to FIG. 53 of U.S. patent application Ser. No. 10/825,900, the contents of which applications are incorporated herein by reference.

FIG. 1 shows common EMI filter circuits such as C, L, PI, etc. It is only the C circuit that has been in common use in cardiac pacemakers to date (U.S. Pat. No. 5,333,095 et. al.). The L₁, L₂, PI, T, LL and 5-Element circuits are desirable low pass circuit configurations for use with either the novel lossy ferrite inductor or cancellation winding technology described herein.

FIG. 2 illustrates attenuation slope curves for various low pass filter circuits. Shown are the attenuation slopes for C, L, PI, T, LL and 5-element EMI filters. As one increases the number of filter elements, the attenuation slope increases. That is, for a given capacitance value, one can achieve a much higher level of EMI attenuation. For MRI applications, particularly desirable configurations include the T or LL. The reason for this is that the added inductance and high frequency resistance also raises the cardiac lead system impedance. Increasing the lead system impedance reduces the MRI currents that circulate in the implanted lead wires. This will substantially reduce undesirable lead wire heating effects.

As can be seen in FIG. 2, there is substantial difference between the single element (feedthrough capacitor or C), the L circuit and the PI circuit configurations. One will notice that the curves become non-linear at lower frequency. Accordingly, if the PI circuit filter is properly designed (so that it does not resonate) it can offer substantially higher attenuation at lower frequencies. As previously mentioned, the slope of the PI circuit is 60 dB per decade. The slope of the L circuit is 40 dB per decade, and the slope of the C circuit is 20 dB per decade.

In FIG. 2, one can see a resonant dip fᵣ in the performance curve of the single element C-section filter. This self-resonance phenomenon is typical of all feedthrough capacitors. Feedthrough capacitor devices resonate far differently than standard monolithic ceramic chip capacitors (MLCCs). In an MLCC, the resonance is caused by parasitic inductance, which in the equivalent circuit, is in series with the capacitor. For an MLCC at resonance, the attenuation actually increases dramatically. However, above resonance the attenuation rapidly falls off as the MLCC capacitor becomes increasingly inductive. The opposite tends to happen in a feedthrough capacitor as illustrated in FIG. 2. This is a more complicated type of parallel transmission line resonance. The feedthrough capacitor continues to function above its self-resonant frequency and is still an effective EMI filter. However, as one can see from the single element C-filter graph of FIG. 2, there is a drop in attenuation at the actual resonant frequency fᵣ. This is undesirable, particularly if the drop in attenuation occurs at the frequency of an EMI emitter such as a cellular telephone. This means that at that particular frequency fᵣ, the implantable medical device, like a cardiac pacemaker, is more susceptible to outside interference. The addition of the inductor slab element not only increases the attenuation slope as shown in FIG. 2, but also minimizes or eliminates the resonant dip phenomenon.

There is another implication to these curves of FIG. 2, which is that one could achieve the same attenuation as the basic feedthrough capacitor, but with a much lower value of capacitance making up for this by the fact that we have series inductance or series resistance elements, which compensate and create a higher attenuation slope.

The tuning process of the present invention involves selecting the proper combinations of much lower value feedthrough capacitors in combination with the series lossy ferrite and/or inductor elements such as to achieve equivalent high frequency EMI attenuation performance. It doesn't even really matter how well the inductor performs in the presence of the MRI field. What is meant by this is that the simple fact that using a feedthrough capacitor that has a lower capacitance value will raise the input impedance of the AIMD. This by itself will greatly reduce the amount of circulating RF current in the AIMD implanted lead wire system. However, in the case of an MRI application, there is a main static field, which can vary anywhere from 0.5 to several Teslas. There are research systems currently in development that even go above 5-Teslas. This main static field can saturate the ferrite core or the magnetic core of most inductive elements. This is not true for an air wound inductor. However, the problem here is that the amount of inductance is very low for the amount of volume required to wind it. In contrast, the lossy ferrite elements and/or iron core inductor elements as described herein, need not work very well (or at all) in the actual bore of an MRI system. This is because when the higher level of EMI filtering is needed, for example, when the patient is in the presence of a microwave oven or a cellular telephone, the lossy ferrite or inductor element will not saturate and will work properly. As shown in FIG. 2, when not in the presence of a biasing field, for example, that from the static field of an MRI system, multi-element filter circuit performance will be achieved. That is, as shown, for example, by the T or LL1, LL2 or 5-element circuits. Accordingly, in order to protect the patient while going about his normal activities, for example, while using a cellular telephone, it is possible to design EMI filter with a very low value of feedthrough capacitance along with correspondingly high values of series inductance and resistance (lossy ferrite) such that the patient will be protected from an EMI point of view. That is, the sensitive electronics or sensing circuits of the AIMD will not malfunction due to this EMI. A typical example would be a cardiac pacemaker patient. It is well documented that EMI can sometimes be sensed as a normal biologic rhythm. This can be catastrophic for a pacemaker dependent patient, in that the pacemaker might interpret EMI as a normal heart beat and inhibit. This means that the pacemaker would shut itself off to save its batteries. In this case, the pacemaker dependent patient would no longer have a heart beat, which is, of course, immediately life threatening. Accordingly, it is very important that a high level of EMI filter attenuation be provided to the patient when said patient is going about his normal daily life activities.

On the other hand, while said patient is under medical supervision undergoing a medical diagnostic or therapy procedure, the same level of EMI filtering is not required. What is more important in this case is that the implanted lead wire system not be subjected to excessive currents which could cause it to overheat and permanently damage surrounding tissues. For example, with cardiac pacemaker patients, it has been demonstrated that after an MRI procedure, surrounding tissue changes and even ablation can occur in the area of the distal Tip. What this means is that there has been some damage to the myocardial tissue. This can result in an increase in the pacemaker capture level (pacing threshold voltage) or even a complete loss of capture. An increase in capture level means that the pacemaker patient would need a much higher voltage output from the pacemaker after the MRI procedure to properly beat the heart as opposed to prior to the procedure. It is highly undesirable and very worrisome for capture level to increase. Not only does increased capture level shorten battery life, but there are concerns about the damage to tissue and the resulting pathology. In certain cases, complete loss of capture has occurred which required life saving procedures followed by implantation of a new pacemaker and associated lead system.

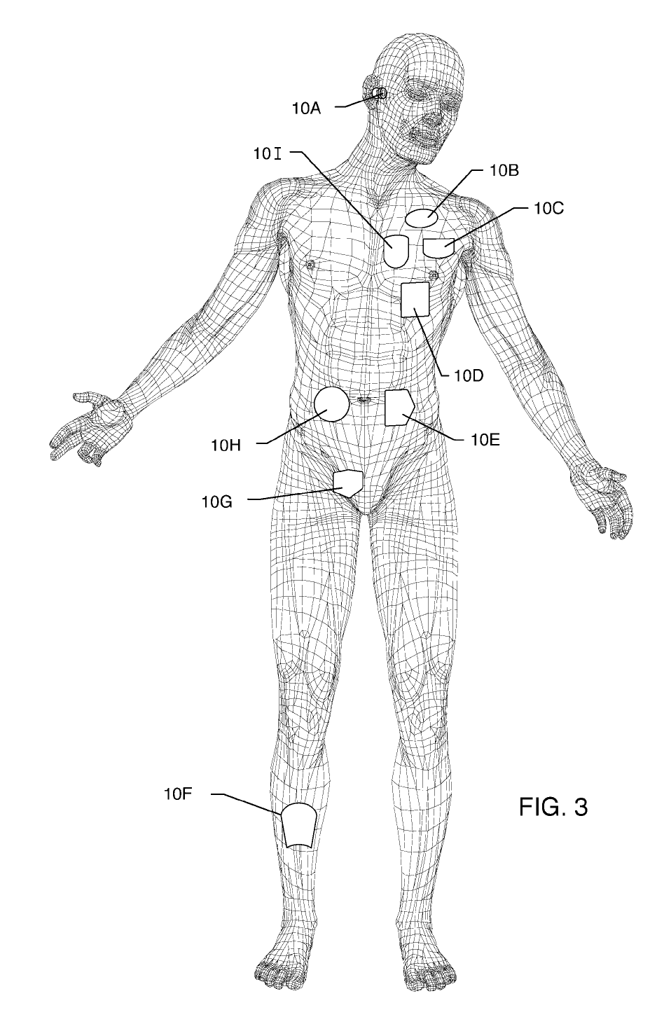

FIG. 3 is an example of the various types of active implantable medical devices 10 currently in use. FIG. 3 is a wire formed diagram of a generic human body showing a number of implanted medical devices. 10A is a family of hearing devices which can include the group of cochlear implants, piezeoelectric sound bridge transducers and the like. 10B includes an entire variety of neurostimulators and brain stimulators. Neurostimulators are used to stimulate the vegas nerve for example to treat epilepsy, obesity and depression. Brain stimulators are similar to a pacemaker-like device and include electrodes implanted deep into the brain for sensing the onset of the seizure and also providing electrical stimulation to brain tissue to prevent the seizure from actually happening. 10C shows a cardiac pacemaker which is well-known in the art. 10D includes the family of left ventricular assist devices (LVAD's), and artificial hearts, including the recently introduced artificial heart known as the Abiocor. 10E includes an entire family of drug pumps which can be used for dispensing of insulin, chemotherapy drugs, pain medications and the like. 10F includes a variety of bone growth stimulators for rapid healing of fractures. 10G includes urinary incontinence devices. 10H includes the family of pain relief spinal cord stimulators and anti-tremor stimulators. Insulin pumps are evolving from passive devices to ones that have sensors and closed loop systems. That is, real time monitoring of blood sugar levels will occur. These devices tend to be more sensitive to EMI than passive pumps that have no sense circuitry. 10H also includes an entire family of other types of neurostimulators used to block pain. 10I includes a family of implantable cardioverter defibrillators (ICD) devices and also includes the family of congestive heart failure devices (CHF). This is also known in the art as cardio resynchronization therapy devices, otherwise known as CRT devices.

Most, if not all, of these AIMDs have associated implanted lead wire systems. Accordingly, there is a concern about overheating literally all of these devices in the presence of medical diagnostic and therapy procedures that involve high levels of RF energy.

It is useful to refer to a 50-ohm system for the purpose of analyzing and tuning the novel passive EMI filter circuit as described herein. There are various circuit simulators, such as P-Spice, which are very useful for this purpose. Using a typical prior art feedthrough capacitor value of 4000 picofarads, we will now analyze the attenuation in decibels (dB) at the various ANSI/AAMI PC69 frequencies defined as 450 MHz to 3 GHz. The following table of values is the 50-ohm attenuation for a 4000-picofarad feedthrough capacitor:

TABLE A

64 MHz (1.5 Tesla MRI frequency) = 32 dB
128 MHz (3 Tesla MRI system) = 38 dB
450 MHz (start of PC69 requirements) = 49 dB
1 GHz > 50 dB
3 GHz (end of PC69 requirement) > 50 dB.

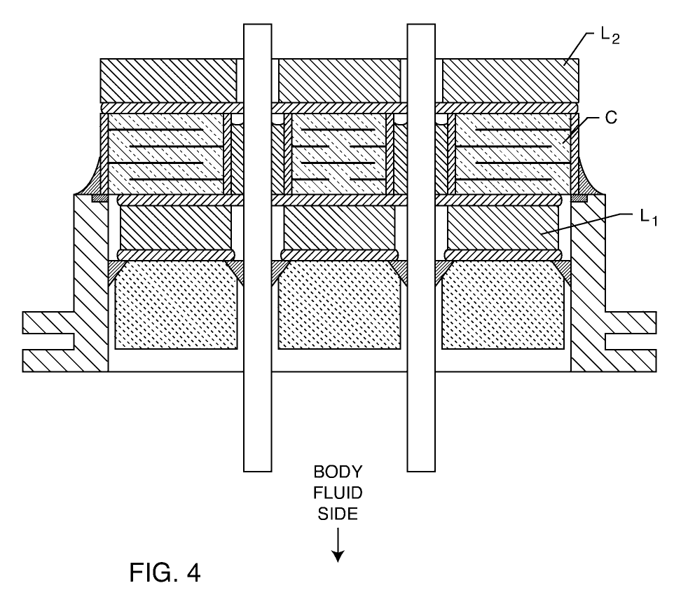

As previously discussed and calculated, the capacitive reactance associated with these high levels of attenuation is 0.31 ohms, which establishes a very low input impedance for the cardiac pacemaker. Now let's perform the same calculations using a T-circuit EMI filter as shown in FIG. 2. This is also better shown in FIG. 4, which is a cross-sectional drawing illustrating a “T” circuit filter configuration. A “T” circuit is also highly efficient in that lossy ferrite inductor L₁ is oriented toward the body fluid side. Lossy ferrite inductor L₂ points toward the electronics of the implantable medical device thereby tending to stabilize the device's input impedance. As previously shown in FIG. 2, the “T” is a very high performance EMI filter that will offer broad attenuation throughout the frequency range from 1 MHz to 100 MHz and above. EMI filters using only a capacitance C, generally are only effective from 100 MHz to about 3 GHz. The “T” section filter as shown in FIG. 4, has all the benefits of a feedthrough capacitor, but with the added benefits of inductances and high frequency dissipative losses placed on both sides of the feedthrough capacitor. The performance of the T filter is not quite as high as the performance of the LL circuit filter, however, it is outstanding compared to all prior art “C” circuit devices.

Referring to FIG. 4, in order to significantly increase the input impedance of the implantable medical device, let us reduce the value of the feedthrough capacitor from 4000 to 400 picofarads. Performing the circuit analysis now becomes more complicated because inductance and resistive properties of the inductor slabs L1 and L2 vary with frequency. For the purposes of the following Table B, these values were determined by materials analyzer measurements at each particular frequency and then plugged (iteratively) into the appropriate circuit simulator program using P-Spice. A typical lossy inductor slab as used in this example has a series inductance of 15.3 nanohenries at 100 MHz and 18.9 nanohenries at 500 MHz, which for these purposes are assumed to be constant up to 3 GHz. The lossy series resistance element of the ferrite slab is 7.3 ohms at 100 MHz, which increases to 12.24 ohms at 500 MHz. Again, the 12.24 ohms is conservatively assumed to be constant up to 3 GHz. For this particular example,

TABLE B
Insertion loss at

| | |
|---|---|
| 64 MHz = | 14.5 dB |
| 128 MHz = | 20.5 dB |
| 450 MHz = | 35 dB |
| 1 GHz = | 49 dB |
| 3 GHz > | 50 dB |

As one can see, there is a compromise with a lower level of insertion loss at the MRI pulsed frequencies, but a high level of attenuation performance in the cellular phone frequency range of 950 MHz and above. By reducing the capacitance value by a factor of 10, one also reduces the current in the induced lead wire system by a factor of 10 due to Ohms Law. Remembering that power is an I²R effect, this reduces the power dissipation in the lead wire of the associated body tissue by a factor of 10² or 100. This results in a very significant reduction in heating in certain sections of the implanted lead wire.

As one can see, this is an iterative or tuning process where one works to reduce the capacitance value as low as possible and still provide compliance to the various regulatory standards and also sufficient attenuation against the RF frequency of the particular piece of medical diagnostic or therapy equipment.

It will be obvious to one skilled in the art that similar procedures apply to every one of the circuits that are described above. This included the L, the PI, the T, the LL, the 5-element and the N element circuit configurations. Each one requires a separate set of calculations and verification (validation) measurements.

Accordingly, it is highly desirable to reduce the capacitance value of the feedthrough capacitor element(s) as much as possible while at the same time providing sufficient EMI filter performance.

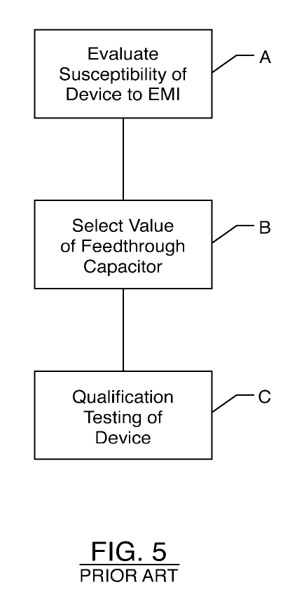

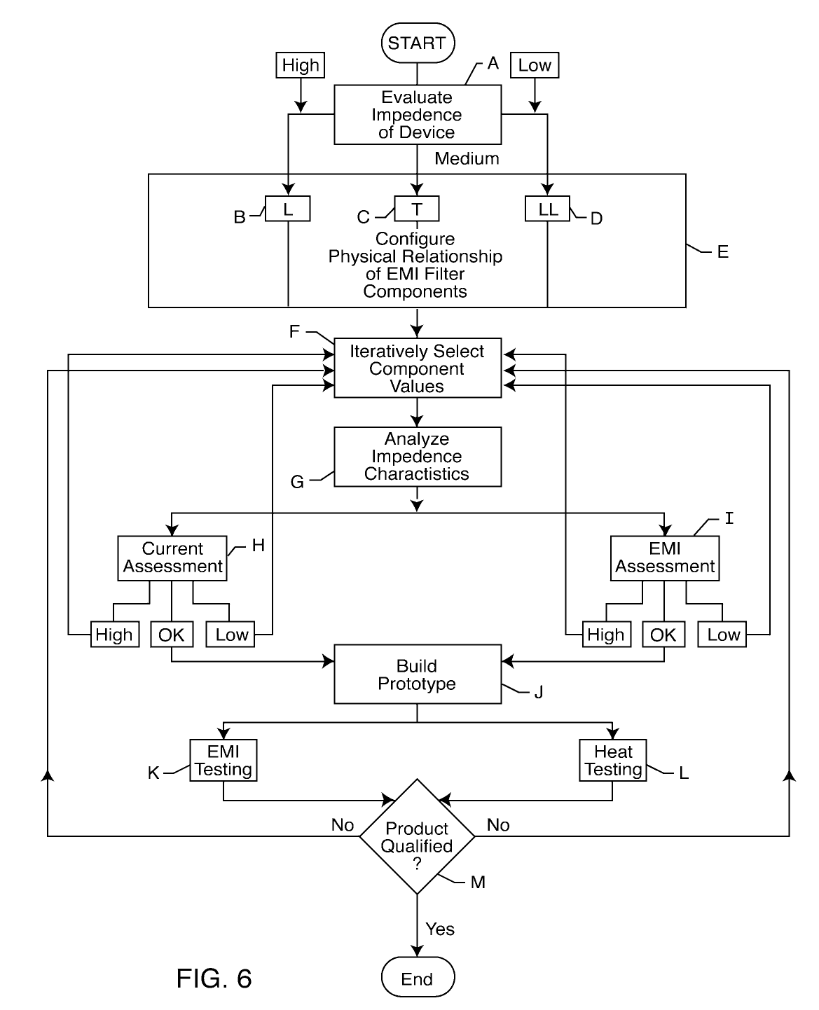

With reference specifically to FIGS. 5 and 6, FIG. 5 is a flow chart illustrating the prior art process of attenuating EMI frequencies with a feedthrough capacitor. Box A illustrates the step of evaluating the susceptibility of the implantable medical device to EMI. This is typically done by testing or by field experience. Box B illustrates the step of selecting a value of feedthrough capacitor. This value is usually the maximum that the circuit can withstand without malfunctioning. For a pacemaker this means the maximum amount of feedthrough capacitance value where leakage back to body tissue would occur. For an implantable defibrillator this is the maximum value of capacitance before the defibrillator pulse degradation would occur. For an implantable defibrillator the capacitance value is generally limited in the range of 1000 to 2000 picofarads. For a cardiac pacemaker the maximum capacitance value is approximately 7800 picofarads. Box C illustrates the step of qualification testing which includes compliance with ANSI/AAMI/PC69. In Europe, equivalent standards include CENELEC or other regulations. As previously mentioned, this process provides a very high degree of EMI filtering but on the downside provides a very low impedance (virtually a short circuit) to MRI RF pulse frequencies. This has the undesirable effect of maximizing the current through implanted lead systems during such medical diagnostic procedures.

FIG. 6 is a flow chart that describes the tuning process of the present invention. Block A is an evaluation of the input impedance of the implantable medical device. This is typically done by using a network analyzer, sophisticated materials analyzer, or spectrum analyzer to look back into the terminals of the active implantable medical device where its implantable leads would normally connect. This is also performed at the MRI RF-Pulse frequencies of interest. For example, for a 1.5 Tesla MRI system which has a pulsed RF frequency of 64 megahertz, this impedance evaluation would be done at 64 megahertz.

Block B shows the step of configuring the physical relationship of the capacitor and the inductor/resistor element of the EMI filter, based on the evaluated input impedance of the AIMD. In the case where the circuits of the implantable medical device look like a very high impedance inherently at 64 megahertz, then one could desirably select an L-Section filter with the capacitor oriented toward the electronics and the lossy ferrite slab or inductor oriented towards the body fluid side of the device (Block C). In the case where the input impedance was medium, for example in the area of 100 to 500 ohms, then a T-Section filter would be more optimum. The second inductor pointing towards the electronics would tend to raise the impedance thereby making the EMI filter circuit tuning more optimum (Block D). In the third case where the internal impedance of the AIMD electronics were quite low (for example: below 50 ohms), then the desirable circuit configuration would be a double L type of filter (Block E). Of course, the L, T and LL circuit configurations are only examples as five element and n-element filters are also possible.

Block F is the process of interatively selecting component values starting with the lowest possible value of capacitance. For example, in prior art pacemakers a typical feedthrough capacitor's value as previously mentioned is 4000 picofarads. In Block F, one would start with a very low value of capacitance (for example, 100 picofarads and then select the maximum value of lossy ferrite inductor slab values that would physically fit the geometry that is available inside the package of the cardiac pacemaker. One would then use a circuit simulator to evaluate the amount of attenuation given the impedance of the cardiac pacemaker that had been previously measured in Block A and then also source impedance in the implanted lead wire system. This step requires using a network analyzer to analyze the impedance for the particular lead wire system (Block G). It will be noted that a unipolar lead has a different impedance than bi-polar leads and spiral leads. Therefore, for the particular active implantable medical devices contemplated, Block G would be the step of taking the lead wire system that is designed to work in conjunction with the AIMD and analyze its impedance characteristic. This is important for the entire circuit simulation as performed in Block F will be more accurate. The circuit simulation that is performed in Block F will allow one to determine the new input impedance for the AIMD and assess whether or not the impedance has been raised sufficiently so that undesirable RF currents that would flow during medical diagnostic procedures and associated heating has been reduced to acceptable levels. At the same time, the circuit simulation will assess the EMI filter attenuation at various high-frequencies to make sure that the AIMD is still adequately protected against environmental emitters (such as cellular phones) and in addition will comply with the various regulatory requirements.

One would first start with an assessment of the current that is induced in the lead wire system (Block H). If the current is found to be too high then one would attempt to reduce the capacitance value even further and use that volume to further increase the amount of lossy ferrite/slab inductance. In the case where the current was very low, one would use the opposite procedures. We could then increase the capacitance value until the current was just able to produce an acceptable level of lead wire heating. This is done at the same time while we're also looking at the EMI attenuation of the filter (Block I). In the case where the EMI attenuation was too low, one would either have to raise the value of the feedthrough capacitor or increase the series of inductive resistance elements until there was an acceptable level of EMI filter attenuation. If the EMI filter performance was too high, this would be an indication that one could further reduce the value of feedthrough capacitor thereby minimizing the current. As a general rule of thumb, the tuning procedure is optimized when you reach an acceptable level of EMI filter attenuation and have the lowest possible value of feedthrough capacitance or capacitors that are consistent with that value. At this point, the design is finalized.

In Block J, one then builds prototypes of the finalized design and then submits them for testing of two types. Testing in Block K is EMI testing in accordance with ANSI/AAMI/PC69 or equivalent standard. Testing in Block L is to expose the system with its associated lead wires in an MRI bore in a Gel Tank and use optical or equivalent Fizo measuring equipment to actually measure the heating in the lead wire system. If both of these levels are acceptable, then the design is deemed qualified. Decision Block M indicates that if both the Blocks K and L testing (in other words for MRI and EMI are both passed) then the product is done and ready for FDA regulatory approval. In the event that the device failed the EMI testing then we would go back up and re-design the filter portion wherein we may have to increase the capacitance value and increase the series inductive elements until we reach an acceptable level of EMI. If we fail the acceptable heating requirement during the MRI board test, then we have to go up and re-design for a lower level of current which would require a lower level of capacitance be designed in. The requirement for this tuning process is that the circuit prediction analysis shown in Block F is not entirely perfect and this is due to the complication as a complex situation involving non-linear impedance interactions and field interactions and various coupling mechanisms that are involved. In the case where both Blocks K and L testing fail, this means that there is a serious design issue which would require re-design of the pacemaker to allow more space. What this means is that we would need more physical room to put in higher levels of inductance or series resistance so that we could further lower the capacitance value.

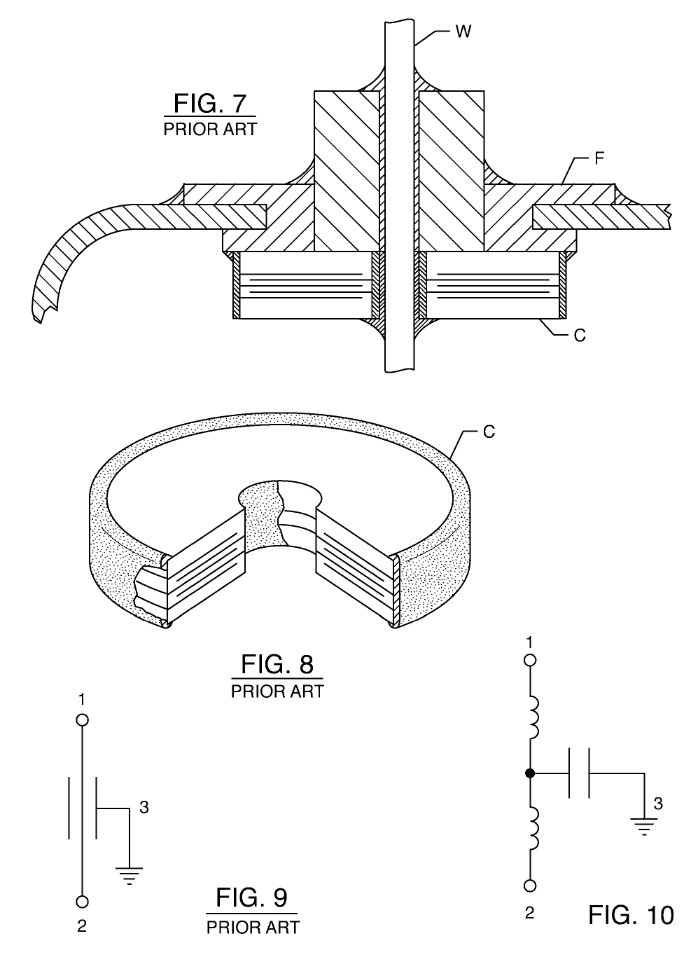

Referring now to FIGS. 7-10, it is also possible to use the inherent lead wire system in the tuning procedure of the present invention. FIGS. 7 and 8 show a prior art unipolar feedthrough capacitor C mounted to the hermetic terminal F of an implantable medical device. By virtue of the principle of physics, the lead wire W has distributed inductance along its entire length. This inductance is relatively small compared to a ferrite core wound inductor. However, an advantage of this inductance is that it will not saturate in the present of the main static field of an MRI machine. FIG. 10 is a modified circuit diagram taken from FIG. 9 of the prior art, which shows these parasitic inductances placed in series. This forms a T-circuit filter similar to that described above. Using the circuit tuning techniques as described herein and accounting for the series inductances, it is possible to slightly reduce the value of the feedthrough capacitor. As previously described herein, it is desirable to keep the value of the feedthrough capacitor as low as practicable to thereby minimize the currents that flow during medical diagnostic and therapy procedures such as MRI. In the prior art, it has been common to use the maximum value of feedthrough capacitor that will fit in the available space and also not to degrade pacemaker or ICD functioning. It is a novel feature of the present invention that the series inductance of the lead wire system be accounted for and incorporated into the design and simulation such that the value of the feedthrough capacitor can be minimized thereby minimizing the currents and thereby the heating that would occur during such RF medical procedures.

Accordingly, it should be apparent that the present invention provides a tuning process for a EMI filter manufactured with passive components for active implantable medical devices wherein in the preferred embodiment:
a passive inductor and/or resistive element is placed in series with the AIMD lead wire at the point of lead wire ingress and egress which is then followed by a parallel feedthrough capacitive element;
the capacitance value of said capacitor is minimized to reduce RF currents in the implantable device lead wire system; and
one or more passive series inductive and/or resistive elements are used to create a multi-element EMI filter that has acceptable attenuation to protect the patient from electromagnetic interference; and
the relative values of the one or more capacitive element(s) which couples implantable device lead wires to an equipotential shield housing are carefully balanced with the passive series components.

The process may be modified:
wherein the series element is an inductor;
wherein the series element is a resistor;
wherein the series element has both inductance and resistance;
where the inductance of lead wires through a feedthrough capacitor provide the series passive elements;
where the series passive element is lossy ferrite inductor slab;
wherein there are a number of possible combinations for capacitors and the series elements which include L, PI, T, LL, 5 element, and N element devices;
wherein the feedthrough capacitive element is placed at the point of lead wire ingress and egress which is then followed by the passive inductor and/or resistive element;
wherein circuit simulation programs are used to carefully balance and trade off the amount of RF current due to diagnostic procedures, imaging or therapy that may be induced in lead wire systems against the amount of EMI filtering required to protect the patient from environmental insults and also pass and comply with certain regulatory standards;
wherein interative EMI and MRI lab testing is used in combination with or in lieu or circuit simulations to tune and optimize EMI filter performance vs. indirect RF current; and
where a combination of simulation and lab work is used.

Although a particular embodiment of the invention has been described in detail for purposes of illustration, modifications may be made without departing from the spirit and scope of the invention. Accordingly, the invention is not to be limited, except as by the appended claims.

What is claimed is:

1. A process for tuning an EMI filter for an active implantable medical device (AIMD), the EMI filter having a capacitor and an inductor/resistor element, comprising the steps of:
evaluating input impedance of the AIMD;
configuring the physical relationship of the capacitor and the inductor/resistor element of the EMI filter, based on the evaluated input impedance of the AIMD;
iteratively selecting component values for the capacitor and inductor/resistor elements of the EMI filter; and
analyzing the impedance characteristics of the selected components through circuit simulation to assess (a) whether the impedance of the EMI filter has been raised sufficiently to reduce undesirable RF currents that would flow during medical diagnostic procedures, and (b) if the AMID is adequately protected against environmental emitters and complies with regulatory requirements.

2. The process of claim 1, including repeating the iteratively selecting and analyzing steps (a) if the impedance of the EMI filter has not been raised sufficiently to reduce undesirable currents that would flow during medical diagnostic procedures, or (b) if the AMID does not adequately protect against environmental emitters and complies with regulatory requirements.

3. The process of claim 1, including the steps of:
building a prototype of the AIMD comprising an EMI filter having selected components that have been assessed to be acceptable;
testing the prototype to determine whether the impedance of the EMI filter has been raised sufficiently to reduce undesirable currents that would flow during medical diagnostic procedures; and
testing the prototype to determine if the AMID is adequately protected against environmental emitters and complies with regulatory requirements.

4. The process of claim 3 including repeating the configuring, iteratively selecting and analyzing steps if the prototype fails either of the testing steps.

5. The process of claim 1, wherein the evaluating step includes the steps of utilizing a network analyzer, sophisticated materials analyzer or spectrum analyzer to look back into the terminal of the AIMD where its implantable leads would normally connect, and performing impedance measurements at RF frequencies of interest.

6. The process of claim 1, wherein the configuring step includes utilizing an inductive/resistive element located at a point of lead wire ingress and egress from the AIMD followed by the capacitor, where the capacitance value of said capacitor is minimized to reduce RF currents in a lead wire system of the AIMD.

7. The process of claim 1, including one or more passive series inductive/resistive elements to create a multi-element EMI filter having acceptable attenuation to protect a patient from electromagnetic interference (EMI).

8. The process of claim 7, wherein the one or more passive series elements comprises an inductor, a resistor, or a combined inductive/resistance element.

9. The process of claim 8, wherein the passive series element comprises a lead wire through the capacitor.

10. The process of claim 8, wherein the series passive element comprises a lossy ferrite inductor slab.

11. The process of claim 7, wherein the passive series element includes an air wound inductor, a chip inductor, a wire wound resistor, a composition resistor, or a toroidal inductor with a ferromagnetic material core.

12. The process of claim 6, wherein the capacitor and the passive series inductive and/or resistive elements are combined to form an L, PI, T, LL, 5-element or N-element device.

13. The process of claim 1, wherein the iteratively selecting step includes the step of selecting a capacitor with a very low value of capacitance and selecting the maximum value of the inductor/resistor element that would physically fit the geometry available inside the package of the AIMD.

14. The process of claim 1, wherein the analyzing step includes the step of utilizing a network or spectrum analyzer to analyze the impedance of lead wire systems associated with the AIMD.

15. The process of claim 1, including the step of optimizing component values of the capacitor and the inductor/resistor elements of the EMI filter such that an acceptable level of attenuation is achieved with the lowest possible value of feedthrough capacitance.

16. A process for tuning an EMI filter for an active implantable medical device (AIMD), the EMI filter having a capacitor and an inductor/resistor element, comprising the steps of:
evaluating input impedance of the AIMD;
configuring the physical relationship of the capacitor and the inductor/resistor element of the EMI filter, based on the evaluated input impedance of the AIMD, by utilizing an inductive/resistive element located at a point of lead wire ingress and egress from the AIMD followed by the capacitor, where the capacitance value of said capacitor is minimized to reduce RF currents in a lead wire system of the AIMD;
iteratively selecting component values for the capacitor and inductor/elements of the EMI filter;
analyzing the impedance characteristics of the selected components through circuit simulation to assess (a) whether the impedance of the EMI filter has been raised sufficiently to reduce undesirable RF currents that would flow during medical diagnostic procedures, and (b) if the AMID is adequately protected against environmental emitters and complies with regulatory requirements;
building a prototype of the AIMD comprising an EMI filter having selected components that have been assessed to be acceptable;
testing the prototype to determine whether the impedance of the EMI filter has been raised sufficiently to reduce undesirable currents that would flow during medical diagnostic procedures; and
testing the prototype to determine if the AMID is adequately protected against environmental emitters and complies with regulatory requirements.

17. The process of claim 16, including the step of optimizing the component values of the capacitor and the inductor/resistor elements of the EMI filter such that an acceptable level of attenuation is achieved with the lowest possible value of feedthrough capacitance.

18. The process of claim 16, including the steps of repeating the iteratively selecting and analyzing steps (a) if the impedance of the EMI filter has not been raised sufficiently to reduce undesirable currents that would flow during medical diagnostic procedures, or (b) if the AMID does not adequately protect against environmental emitters and complies with regulatory requirements, and repeating the configuring, iteratively selecting and analyzing steps if the prototype fails either of the testing steps.

19. The process of claim 16, wherein the evaluating step includes the steps of utilizing a network analyzer, sophisticated materials analyzer or spectrum analyzer to look back into the terminal of the AIMD where its implantable leads would normally connect, and performing impedance measurements at RF frequencies of interest.

20. The process of claim 16, including one or more passive series inductive/resistive elements to create a multi-element EMI filter having acceptable attenuation to protect a patient from electromagnetic interference (EMI).

21. The process of claim 20, wherein the one or more passive series elements comprises an inductor, a resistor, a combined inductive/resistance element, an air wound inductor, a chip inductor, a wire wound resistor, a composition resistor, or a toroidal inductor with a ferromagnetic material core.

22. The process of claim 20, wherein the passive series elements comprises a lead wire through the capacitor or a lossy ferrite inductor slab.

23. The process of claim 16, wherein the capacitor and the passive series inductive and/or resistive elements are combined to form an L, PI, T, LL, 5-element or N-element device.

24. The process of claim 16, wherein the iteratively selecting step includes the step of selecting a capacitor with a very low value of capacitance and selecting the maximum value of the inductor/resistor element that would physically fit the geometry available inside the package of the AIMD.

25. The process of claim 16, wherein the analyzing step includes the step of utilizing a network or spectrum analyzer to analyze the impedance of lead wire systems associated with the AIMD.
